(19) United States
(12) Patent Application Publication
(10) Pub. No.: US 2014/0364867 A1
(43) Pub. Date: Dec. 11, 2014

(54) CIRCUMCISION DEVICE

(71) Applicants: Kuang Ling Siow, Johor (MY); Kuang Choong Siew, Johor (MY)

(72) Inventors: Kuang Ling Siow, Johor (MY); Kuang Choong Siew, Johor (MY)

(73) Assignee: MEDIGLOBAL ENTERPRISE SDN BHD

(21) Appl. No.: 14/351,542

(22) PCT Filed: Aug. 29, 2012
(86) PCT No.: PCT/MY2012/000244

§ 371 Date: Aug. 22, 2014

(30) Foreign Application Priority Data
Oct. 12, 2011 (MY) PI 2011004889

(51) Int. Cl. A61B 17/326

(52) U.S. Cl. CPC A61B 17/326
USPC 606/118

(57) ABSTRACT

The present invention relates to a device for circumcising a penis characterised by: a primary ring (100) including an anterior end (101), a posterior end (102), and a plurality of indentations (103) on the anterior end; a tubular member (200) including a plurality of protrusions (201) for maintaining a foreskin of the penis in a proximal position; wherein the tubular member (200) is configured to be received by the anterior end (101) of the primary ring (100); a locking ring (300) including a plurality of projections (301) configured to engage with the 300 plurality of indentations (103) for securing the foreskin against the primary ring (100); and a cover ring (400) for tightening the locking ring (300) on the primary ring (100).

### BACKGROUND OF THE INVENTION

1. Field of the Invention

This invention relates to a device for circumcising a penis, and more particularly to a device for circumcising a penis without wound and cutting.

2. Description of Related Arts

The procedure of circumcision usually involves surgical removal of some or the entire foreskin from the penis. The removal of redundant foreskin inevitably leads to bleeding, pain and hospitalization. In some part of the world, some are still using surgical scissors to cut the foreskin. The trimming obtained is usually not only crude and rough but also causes bleeding and needs sutures to be applied to the cut edges. Generally, sterilized gauze and dressing are also applied and sometimes antibiotics may have to be prescribed to prevent any further infection.

In some situation, patient is unable to walk and feel intolerably painful at each change of dressing after operation. The patient may also suffer great pain in the end at removal of stitches. Hence, there is a need to have a circumcision procedure that is less painful and suffering to the patient.

In many situations, laser and high-frequency electric-knife technology have been used in foreskin circumcision, they have only substituted scissors cutting and solidified the bleeding spot. Such a therapeutic method is likely to subject the patient to tissular burn and infection, while the expenditure for infection prevention is very high.

Conventionally, there have been a number of instruments known for performing circumcision operations. One of the occlusion techniques that have been introduced is the plastibell technique. Plastibell technique uses a plastic bell with a groove at the back of the plastic bell which is slipped between glans and foreskin of the penis. The foreskin is pulled slightly forward and suture material is looped around in the groove and a surgical knot is tied tightly. This technique is still using surgical knot which may cause the patient to feel pain during the procedure. Besides that, there is a risk in the technique as there is a possibility that the knot becomes too tight or the foreskin is pulled too tightly that it may cause the plastic bell to dig into the glans and obstructing the urethra.

U.S. Pat. No. 5,860,988 disclosed a device for performing circumcisions comprising two interlockable rings, a larger outer ring which has a continuous groove or channel within its circumference, and a smaller inner ring which has a solid circumference and is sized to fit into the groove or channel of the outer ring so that the two rings from a locking device on the penis which clamps the foreskin of the penis into a fixed position. A surgeon will need to perform incision to remove the excess foreskin and increase the likelihood of injury to the patient. Hence, it may be a painful procedure and patient will need to stay in hospital after the procedure.

US Publication No. 2010/0114112 A1 disclosed a circumcision clamp and surgical kit including a rod having a bell-shaped member at a first end for receiving the head of a penis and an engagement structure at an opposing end; a base including two hinged arms, a lever, and an adjustment structure. Soon after the circumcision has been performed, the clamp is dismantled and removed. The exposed cut edges generally need to be sutured and aseptic dressing has to be applied. The wound has to be closely watched for infection, bleeding and oozing for the next few days. Hence, the use of circumcision clamp leads to a painful procedure and may cause complications to patient if the surgeon is not well-trained.

U.S. Pat. No. 3,612,057 disclosed a circumcision clamp comprising an uninterrupted or solid inner ring adapted to be engaged under the foreskin, and a concentric, split outer ring integrally connected to the inner ring by means of a generally radially oriented pillar or web, the outer ring being engageable over the foreskin and the connecting pillar or web being disposed in the usual dorsal slit in the foreskin, and the outer ring being constrictable into clamping position about the inner ring. However, the circumcision clamp may not achieve an “instant occlusion” at the site of clamping. There is possibility of injuring glans of the penis for such a device like the cited patent during circumcision procedure as the glans is not protected. In some circumstances, the patient might be able to unlock the clamp and therefore, the procedure becomes incomplete.

Malaysian Patent PI20090291 disclosed a disposable circumcision device designed to avoid complications associated therewith. The drawback of the cited patent is that it requires the use of a fastener and a surgical blade which may lead to unintentional injury during the procedure. Though, the cited patent suggested the procedure leaves no open wound, the use of surgical blade in the beginning may affect the patient psychologically. This Malaysian patent application P120090291 is hereby incorporated by reference in its entirety.

Accordingly, it can be seen in the prior arts that there exists a need to provide a circumcision device without undergoing surgery procedure avoiding medical complications and reduce pain and likelihood of injury to a patient during a circumcision procedure.

### SUMMARY OF INVENTION

It is an objective of the present invention to provide a circumcision device that reduces the likelihood of injury to a patient during a circumcision procedure.

It is also an objective of the present invention to provide a disposable circumcision device.

It is yet another objective of the present invention to provide a circumcision device that requires no surgical procedure or knotting and leaves no wound after the circumcision procedure.

It is a further objective of the present invention to provide a circumcision device that able to perform the circumcision procedure faster and efficient without having the patient staying in hospital.

Accordingly, these objectives may be achieved by following the teachings of the present invention. The present invention relates to a device for circumcising a penis characterised by: a primary ring including an anterior end, a posterior end, and a plurality of indentations on the anterior end; a tubular member including a plurality of protrusion for maintaining a foreskin of the penis in a proximal position; wherein the tubular member is configured to be received by the anterior end of the primary ring; a locking ring including a plurality of projections configured to engage with the plurality of indentations for securing the foreskin against the primary ring; and a cover ring for tightening the locking ring on the primary ring.

### BRIEF DESCRIPTION OF THE DRAWINGS

The features of the invention will be more readily understood and appreciated from the following detailed description when read in conjunction with the accompanying drawings of the preferred embodiment of the present invention, in which:

FIG. 1 is a drawing showing an exploded view of a device for circumcising a penis;

FIG. 2a is an isometric drawing showing the device in assembled mode;

FIG. 2b is a drawing showing a side view of the device in assembled mode;

FIG. 2c is an isometric drawing showing the device in assembled mode;

FIG. 3a is a drawing showing a side view of a primary ring;

FIG. 3b is an isometric drawing showing the primary ring;

FIG. 3c is a posterior drawing showing the primary ring;

FIG. 3d is an anterior drawing showing the primary ring;

FIG. 3e is an isometric drawing showing the primary ring;

FIG. 4a is an isometric drawing showing a cover ring with a plurality of teeth circumferentially defined externally around the cover ring;

FIG. 4b is an isometric drawing showing the cover ring with the plurality of teeth circumferentially defined internally around the cover ring;

FIG. 4c is an isometric drawing showing the cover ring without the plurality of teeth;

FIG. 5a is a drawing a side view of a tubular member;

FIG. 5b is an isometric drawing showing the tubular member;

FIG. 6a is posterior drawing showing a locking ring;

FIG. 6b is an anterior drawing showing the locking ring;

FIG. 6c is a drawing showing a side view of the locking ring;

FIG. 6d is a posterior drawing showing the locking ring; and

FIG. 7 is a drawing showing the assembly of the device for circumcising a penis.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting but merely as a basis for claims. It should be understood that the drawings and detailed description thereto are not intended to limit the invention to the particular form disclosed, but on the contrary, the invention is to cover all modification, equivalents and alternatives falling within the scope of the present invention as defined by the appended claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the words “a” or “an” mean “at least one” and the word “plurality” means one or more, unless otherwise mentioned. Where the abbreviations of technical terms are used, these indicate the commonly accepted meanings as known in the technical field. For ease of reference, common reference numerals will be used throughout the figures when referring to the same or similar features common to the figures.

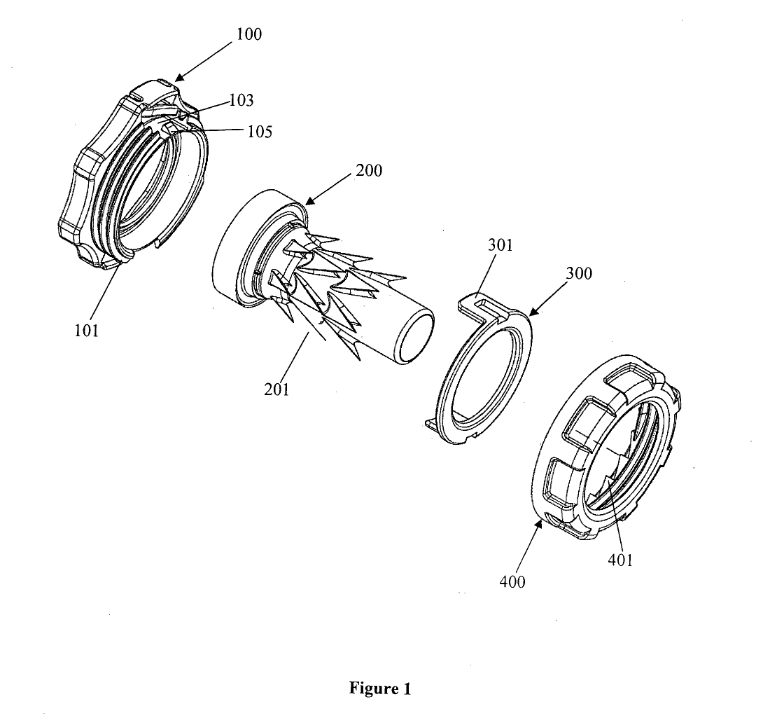

The present invention will now be described with reference to FIGS. 1-7.

The present invention as shown in FIGS. 1 relates to a device for circumcising a penis characterised by:
a primary ring (100) including an anterior end (101), a posterior end (102), and a plurality of indentations (103) on the anterior end;
a tubular member (200) including a plurality of protrusions (201) for maintaining a foreskin of the penis in a proximal position;
wherein the tubular member (200) is configured to be received by the anterior end (101) of the primary ring (100);
a locking ring (300) including a plurality of projections (301) configured to engage with the plurality of indentations (302) for securing the foreskin against the primary ring (100); and
a cover ring (400) for tightening the locking ring (300) on the primary ring (100).

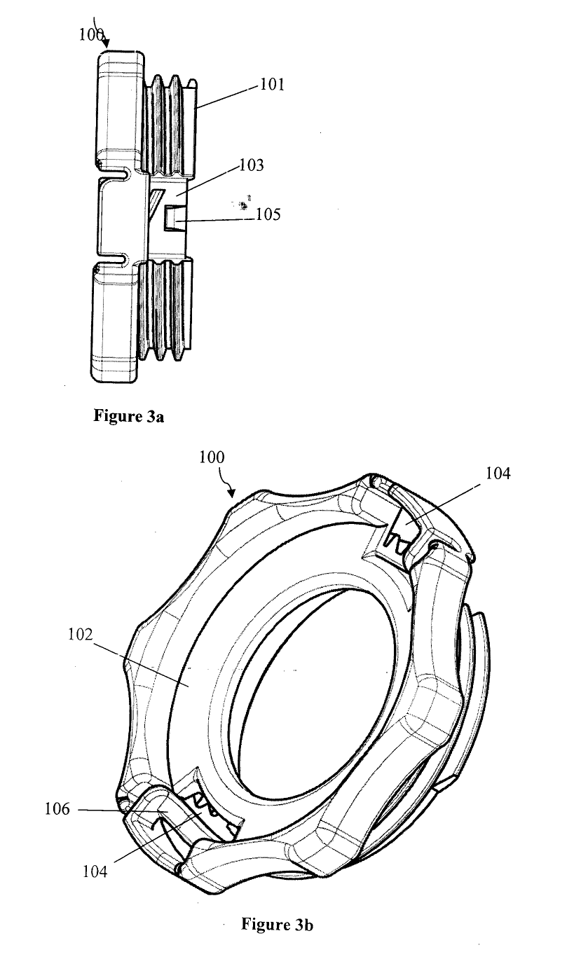

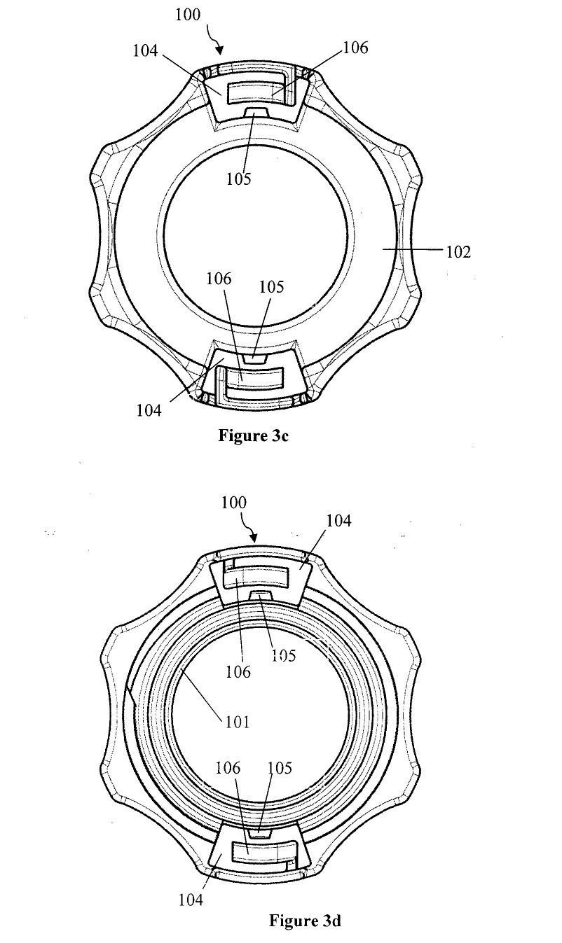

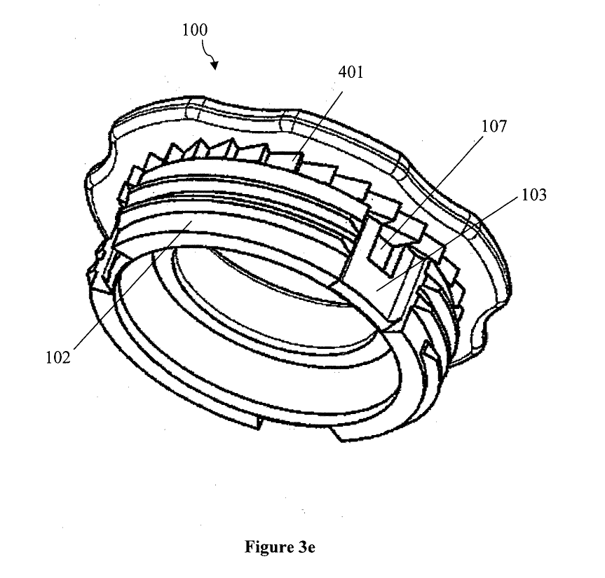

In a preferred embodiment of the device for circumcising the penis, the anterior end (101) is externally threaded (referring to FIG. 3a).

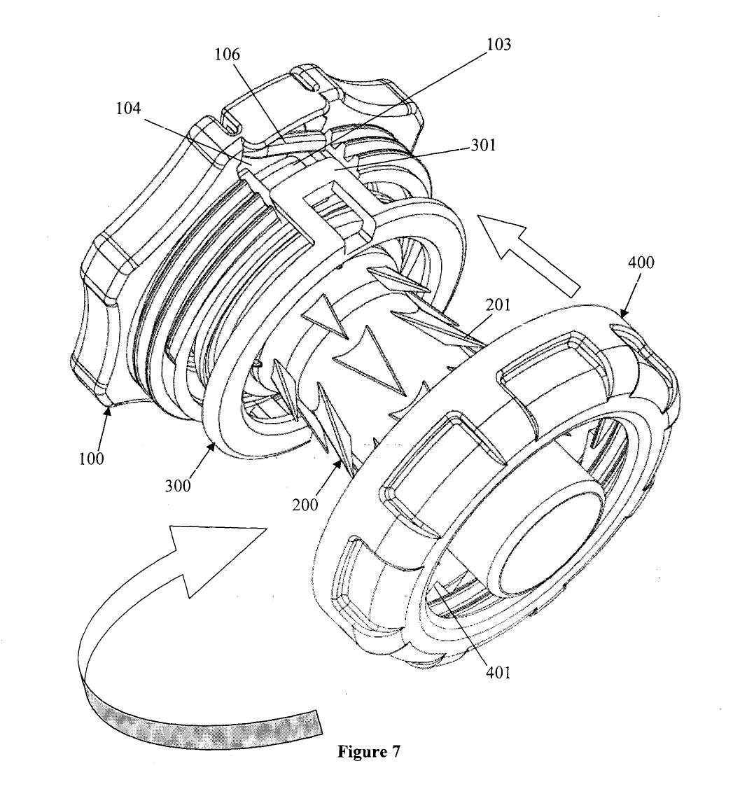

In a preferred embodiment of the device for circumcising the penis, the posterior end (102) of the primary ring (100) further comprises a plurality of apertures (104), as shown in FIG. 7, for receiving the plurality of projections (301) of the locking ring (300).

In a preferred embodiment of the device for circumcising the penis, the primary ring (100) has a tear-away tab (106) on the posterior end (102) to allow the locking ring (300) to be disengaged from the primary ring (100) (referring to FIGS. 3c-3d).

In a preferred embodiment of the device for circumcising the penis, the primary ring (100) has a plurality of teeth (401) circumferentially defined around said primary ring (100) to prevent the disengagement of the cover ring (400) from the primary ring (100) (referring to FIG. 3e).

In a preferred embodiment of the device for circumcising the penis, each of the plurality of indentations (103) has a stopper (105) to prevent the removal of the locking ring (300) from the primary ring (100).

In a preferred embodiment of the device for circumcising the penis, the plurality of indentations (103) has a groove (107) to engage with the plurality of projections (301) of the locking ring (300).

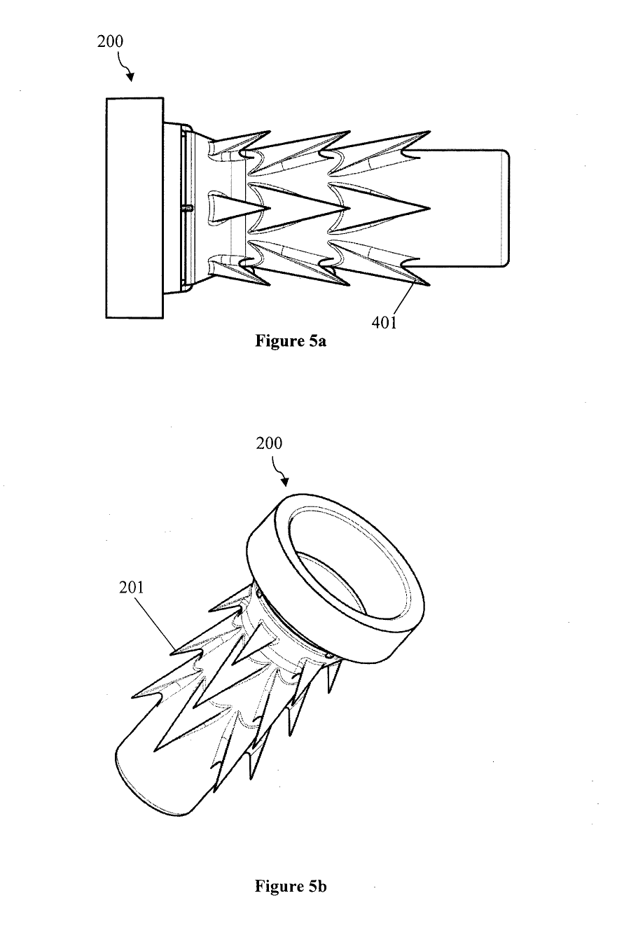

In a preferred embodiment of the device for circumcising the penis, the plurality of protrusions (201) comprises V-shaped protrusions (referring to FIG. 5a-5b).

The plurality of protrusions (201) maintains the foreskin of the penis in a proximal position.

In a preferred embodiment of the device for circumcising the penis, each of the plurality of projections (301) has a stopper (105) to prevent the removal of the locking ring (300) from the primary ring (100).

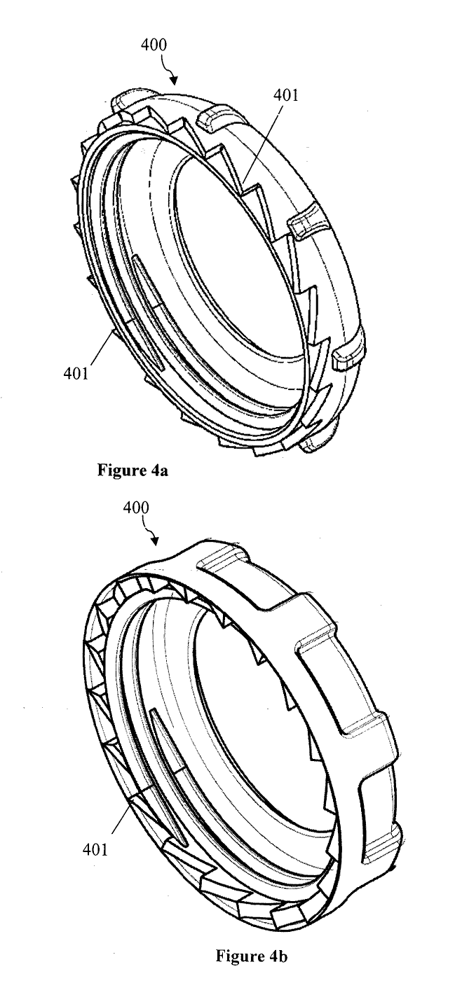

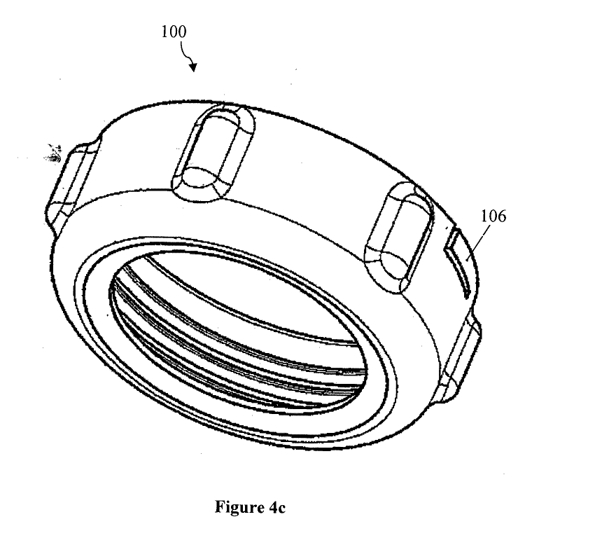

In a preferred embodiment of the device for circumcising the penis, the cover ring (400) is internally threaded (referring to FIGS. 4a-4b).

In a preferred embodiment of the device for circumcising the penis, the cover ring (400) has a plurality of teeth (401) circumferentially defined internally around said cover ring (400) (referring to FIG. 4b) to prevent the device from being disassembled.

In a preferred embodiment of the device for circumcising the penis, the cover ring (400) includes the plurality of teeth (401) circumferentially defined externally around said cover ring (400) (referring to FIG. 4a) to prevent the device from being disassembled.

In a preferred embodiment of the device for circumcising the penis, the cover ring (400) is screwed on to the primary ring (100) to constrict blood flow to the foreskin.

In a preferred embodiment of the device for circumcising the penis, the primary ring (100), the locking ring (300), and the cover ring (400) remains on the penis for at least five days after a procedure for circumcising the penis.

In a preferred embodiment of the device for circumcising the penis, the foreskin, the primary ring (100), the locking ring (300), and the cover ring (400) fall off from penis after a minimum of five days after a procedure for circumcising the penis.

### Method of Using the Present Invention

A preferred method of using the device of the present invention for circumcising the penis comprises the steps of:
inserting the primary ring (100) over the penis;
inserting the penis into the tubular member (200);
pulling the primary ring (100) over to the tubular member (200), simultaneously pulling the foreskin slightly forward over the plurality of protrusions (201) to maintain the foreskin at a proximal position, wherein the anterior end (101) of the primary ring (100) receives the tubular member (200);
pushing the locking ring (300) over the tubular member (200) and foreskin towards the primary ring (100) for locking the foreskin against the primary ring (100), wherein the plurality of projections (301) of the locking ring (300) engage with the plurality of indentations (103) on the anterior end (101) of the primary ring (100);
screwing the cover ring (400) over the locking ring (300) to the anterior end (101) of the primary ring (100) to clamp the foreskin against the primary ring (100);
wherein the tubular member (200) is removed thereafter; and
wherein the tightening of the cover ring (400) and the locking ring (300) on the foreskin of the penis against the primary ring (100) restricts blood flow to the foreskin, thus causing the foreskin to wither and drop off with the primary ring (100), the locking ring (300), and the cover ring (400) after a few days.

As the cover ring (400) tightens the locking ring (300) on the primary ring (100) to clamp the foreskin against the primary ring (100), the device for circumcising penis of the present invention involves no cutting or tying with thread. As the blood flow to the foreskin is restricted due to the tightening of the cover ring (400) on the primary ring (100), the foreskin withers and therefore drops off with the primary ring (100), the locking ring (300), and the cover ring (400) after a few days. This ensures a woundless circumcision procedure and allows the patient to wear trousers after the circumcision procedure.

### Primary Ring (100)

In a preferred embodiment, the primary ring (100) has sharp edges around the anterior end (101) to ensure blood supply to the foreskin of the penis is restricted. The sharp edges herein are defined as having an acute or cutting edge sufficient to cut into the foreskin of the penis. Ina n alternative embodiment, the primary ring (100) may not have the sharp edges as it is also possible to sufficiently restrict blood supply to the foreskin by the tightening of the primary ring (100), locking ring (300), and tubular member (200) against the foreskin.

In the present invention, the primary ring (100) is to be positioned firstly over the penis. The primary ring (100) may be provided in different circumference size depending on the size of the penis.

In a preferred embodiment as shown in FIGS. 3c-3d, the primary ring (100) has a tear away tab (106) to allow disengaging of the locking ring (300) from the primary ring (100). The tab (106) can be torn away to free the locking ring (300).

In a preferred embodiment of the primary ring (100) as shown in FIG. 3c-3d, the tab (106) allows removal of the locking ring (300) from the primary ring (100). It may be desirable to remove the locking ring (300) from the primary ring (100) under certain conditions, for example if unintentional injury on the penis occurs during the circumcision procedure or if either the primary ring (100) or the tubular member (200) is positioned wrongly on the penis. In a preferred embodiment, the tab (106) can be torn away if the cover ring (400) has not yet been screwed on the primary ring (100).

FIG. 3e shows an example of the primary ring (100) wherein the primary ring (100) comprises a plurality of teeth (401) circumferentially defined around the primary ring (100) to prevent the disengagement of the cover ring (400) from the primary ring (100). In an example shown in FIG. 3e, the plurality of indentations (103) of the primary ring (100) has no stopper (105) but has the groove (107) to receive the plurality of projections (301) of the locking ring (300).

### Tubular Member (200)

In a preferred embodiment, the tubular member (200) has sharp edges to ensure blood supply to the foreskin of the penis is restricted. The sharp edges herein are defined as having an acute or cutting edge sufficient to cut into the foreskin of the penis. Ina n alternative embodiment, the tubular member (200) may not have the sharp edges as it is also possible to sufficiently restrict blood supply to the foreskin by the tightening of the primary ring (100), locking ring (300), and tubular member (200) against the foreskin.

Upon the insertion of the penis in the tubular member (200), the foreskin is pulled slightly forward over the plurality of protrusions (201). The tubular member (200) will be received into the anterior end (101) of the primary ring (100). Then, the locking ring (300) is placed over the foreskin. It is preferable to have the plurality of protrusions (201) to hold the foreskin in the proximal position while the locking ring (300) is pushed straight towards the primary ring (100).

As the penis is inserted into the tubular member (200), the tubular member (200) is able to protect the glans of the penis during circumcision procedure, thus reducing patient's anxiety during the procedure. The tubular member (200) may be removed after the cover ring (400) is screwed to the primary ring (100) at a later stage of the circumcision procedure. It is not necessary to keep the tubular member (200) on the penis as the tubular member (200) works to hold the foreskin in place during the circumcision procedure and to protect the glans from unintentional injury.

In a preferred embodiment, the tubular member (200) is slidably movable on the penis and is not externally or internally threaded (referring to FIGS. 5a and 5b). The tubular member (200) is preferably slidably movable so that the tubular member (200) can be easily received by the primary ring (100). Once the foreskin is tightened on the primary ring (100) by the locking ring (300) and the cover ring (400), the tubular member (200) can be removed by sliding it out from the penis.

Preferably, the tubular member (200) is not screwed or tightened to any other element of the circumcision device. Nonetheless, it would be obvious to the person skilled in the art that the tubular member (200) may be modified by providing thread on any part of the tubular member (200). Thus the tubular member (200) may be screwed on to another element of the circumcision device to temporarily secure said tubular member (200).

### Locking Ring (300)

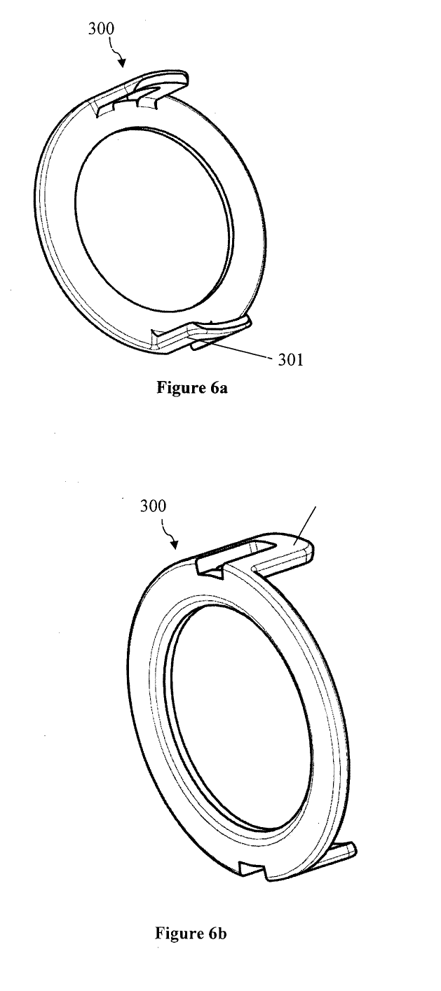

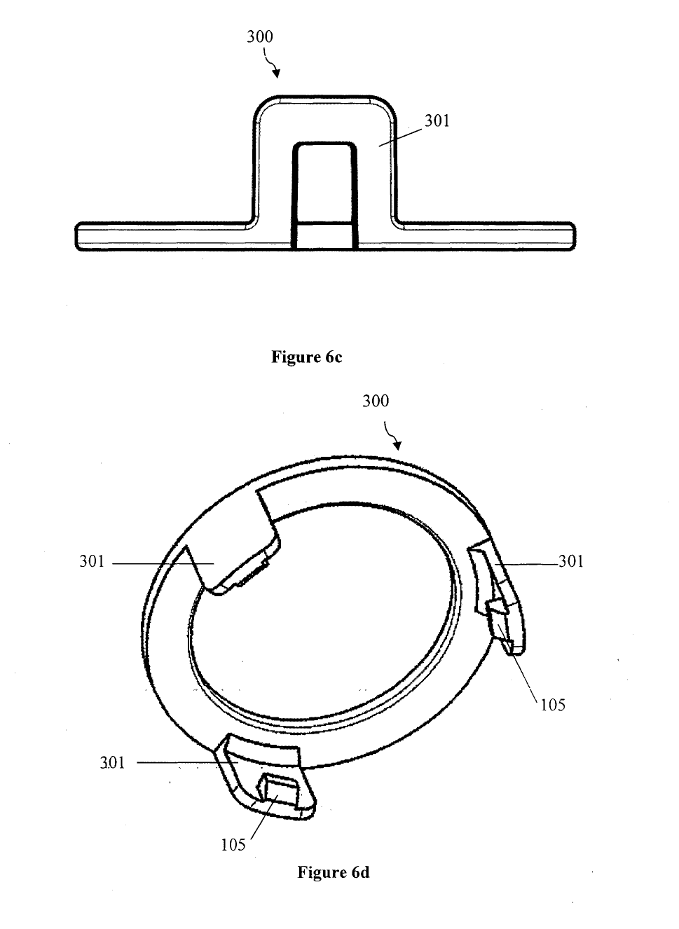

FIGS. 6a-6c show the locking ring. Referring to FIGS. 6a-6b, the plurality of projections (301) preferably comprises two projections. FIG. 6d shows that the plurality of projections (301) preferably comprises three projections.

In a preferred embodiment, the locking ring (300) has sharp edges around the locking ring (300) or the plurality of projections (301) to ensure blood supply to the foreskin of the penis is restricted. The sharp edges herein are defined as having an acute or cutting edge sufficient to cut into the foreskin of the penis. Ina n alternative embodiment, the locking ring (300) may not have the sharp edges as it is also possible to sufficiently restrict blood supply to the foreskin by the tightening of the primary ring (100), locking ring (300), and tubular member (200) against the foreskin.

The plurality of projections (301) of the locking ring (300) may have different shapes and designs. For example, as shown in FIG. 6c, one of the plurality of projections (301) is in a rectangular-shaped-ring, which will engage with one of the plurality of indentations (103) of the primary ring (100). Another example is shown in FIG. 6d, wherein the plurality of projections (301) is in a rectangular shaped projection having the stopper (105) to complement the plurality of indentations (103).

The plurality of indentations (103) of the primary ring (100) complements the plurality of projections (301) of the locking ring (300). During the circumcision procedure, the locking ring (300) is pushed straight over the tubular member (200) and towards the primary ring (300). The locking ring (300) also prevents the penis from twisting when the cover ring (400) is screwed to the primary ring (100).

In a preferred embodiment, the engagement of the plurality of indentations (103) with the plurality of projections (301) forms a locking mechanism. The locking mechanism may be irreversible wherein after the engagement of the plurality of projections (301) on the plurality of indentations (103), the plurality of projections (301) cannot be removed from the primary ring (100). This ensures that the device of the present invention is not reusable thus maintaining hygiene of the device.

For example, as shown in FIGS. 3e and 6d, the stopper (105) of the plurality of projections (301) may rest on the groove of the plurality of indentations (103) of the primary ring (100).

Another example, as shown in FIG. 3c-3d, the plurality of indentations (103) has the stopper (105) which engages with the plurality of projections (301) (referring to FIG. 7). The stopper (105) will prevent the removal of the locking ring (300) from the primary ring (100). Said stopper (105) can be modified by any person skilled in the art for a purpose of locking the plurality of projections (301) to the plurality of indentations (103).

While the present invention is preferably a disposable device, it is also possible to provide an embodiment of the present invention that is reusable. For example, the stopper (105) may be excluded in an alternate embodiment, thus allowing the locking ring to be removed from the primary ring (100).

### Cover Ring (400)

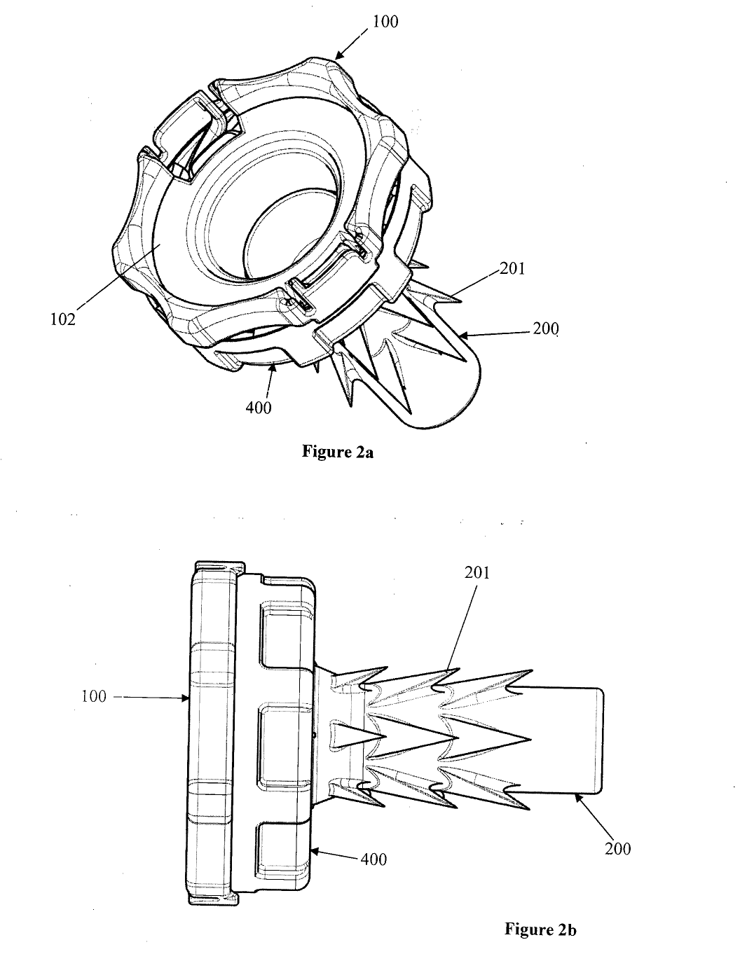

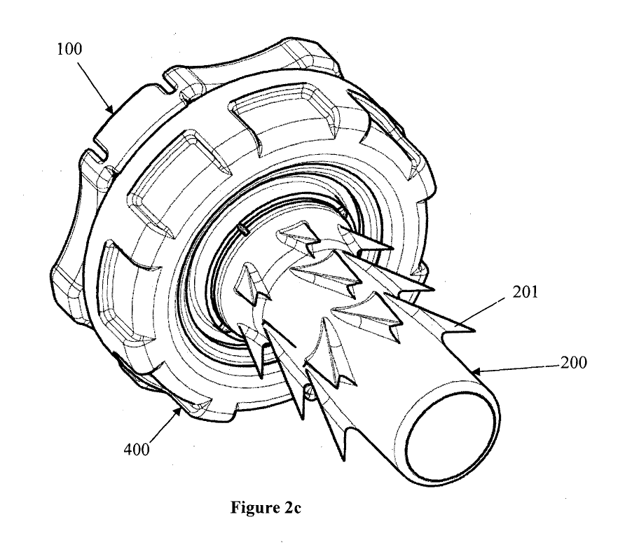

In a preferred embodiment of the present invention, the internally threaded cover ring (400) is screwed on the externally threaded anterior end (101) of the primary ring (100) (referring to FIGS. 2a-2c, and FIG. 7).

In a preferred embodiment of the present invention, the plurality of teeth (401) can be circumferentially defined either internally or externally around the cover ring (400) for tightening and securing the locking ring (300) on the primary ring (100). The plurality of teeth may act as a locking mechanism to prevent the device from being disassembled (referring to FIG. 4a-4b). The plurality of teeth (401) make it impossible to disassemble the device as the plurality teeth only allows screwing of the cover ring (400) on the primary ring (100) at one direction. For example in FIG. 7, the screwing of the cover ring (400) is made in a clock-wise direction and thus the cover ring (400) cannot be unscrewed anti-clock wise due to the slant feature of the plurality of teeth (401) as shown in FIG. 4a. Said feature of the plurality of teeth (401) prevents the removal of the cover ring (400) from the primary ring (100) thus making the device not reusable. Therefore, it is also possible to provide an embodiment of the present invention that is reusable by removing the plurality of teeth (401) as shown in FIG. 4c.

In a preferred embodiment as shown in FIG. 4c, the cover ring (400) has a tab (106) to allow disengaging of the cover ring (400) from the primary ring (100). In the preferred embodiment, the tab (106) can be torn away.

After the screwing of the cover ring (400) on the primary ring (100), the tubular member (200) can be removed. Once the blood supply is restricted, the foreskin will wither and drop off. In a preferred embodiment of the present invention, the foreskin will drop off with the primary ring (100), the locking ring (300), and the cover ring (400) after a minimum of five days after a procedure for circumcising the penis.

FIG. 7 shows the assembly of the elements for circumcising device. FIG. 2a-2c shows how the device would look like on a penis after the assembly of the device for circumcising device.

The device for circumcising penis of the present invention may be provided in different sizes depending on the size of the penis.

Below is example of combination of different parts of the circumcision device from which the advantages of the present invention may be more readily understood. It is to be understood that the following example is for illustrative purpose only and should not be construed to limit the present invention in any way.

### Example A

In one exemplary embodiment, the circumcision device of the present invention comprises the primary ring (100) shown in FIG. 3, the tubular member (200) shown in FIG. 5a, the locking ring (300) shown in FIG. 6a, and the cover ring (400) shown in FIG. 4b.

### Example B

In another exemplary embodiment, the circumcision device of the present invention comprises the primary ring (100) shown in FIG. 3e, the tubular member (200) shown in FIG. 5a, the locking ring (300) shown in FIG. 6d, and the cover ring shown in FIG. 4a.

Although the present invention has been described with reference to specific embodiments, also shown in the appended figures, it will be apparent for those skilled in the art that many variations and modifications can be done within the scope of the invention as described in the specification and defined in the following claims.

Description of the reference numerals used in the accompanying drawings according to the present invention:

| Reference Numerals | Description |
|---|---|
| 100 | Primary ring |
| 101 | Anterior end |
| 102 | Posterior end |
| 103 | Plurality of indentations |
| 104 | Plurality of apertures |
| 105 | Stopper |
| 106 | Tab |
| 107 | Groove |
| 200 | Tubular member |
| 201 | Plurality of protrusions |
| 300 | Locking ring |
| 301 | Plurality of projections |
| 400 | Cover ring |
| 401 | Plurality of teeth |

I/We claim:

1. A device for circumcising a penis characterised by:
a primary ring (100) including an anterior end (101), a posterior end (102), and a plurality of indentations (103) on the anterior end (101);
a tubular member (200) including a plurality of protrusions (201) for maintaining a foreskin of the penis in a proximal position;
wherein the tubular member (200) is configured to be received by the anterior end (101) of the primary ring (100);
a locking ring (300) including a plurality of projections (301) configured to engage with the plurality of indentations (103) for securing the foreskin against the primary ring (100); and
a cover ring (400) for tightening the locking ring (300) on the primary ring (100).

2. A device for circumcising penis, according to claim 1, wherein the anterior end (101) is externally threaded.

3. A device for circumcising penis, according to claim 1, wherein the posterior end (102) of the primary ring (100) further comprises a plurality of apertures (104) for receiving the plurality of projections (301) of the locking ring (300)

4. A device for circumcising penis, according to claim 1, wherein the primary ring (100) has a tear-away tab (106) on the posterior end (102) to allow the locking ring (300) to be disengaged from the primary ring (100).

5. A device for circumcising penis, according to claim 1, wherein the primary ring (100) has a plurality of teeth (401) circumferentially defined around said primary ring (100) to prevent the disengagement of the cover ring (400) from the primary ring (100).

6. A device for circumcising penis, according to claim 1, wherein each of the plurality of indentations (103) has a stopper (105) to prevent the removal of the locking ring (300) from the primary ring (100).

7. A device for circumcising penis, according to claim 1, wherein the plurality of indentations (103) has a groove (107) to engage with the plurality of projections (301) of the locking ring (300).

8. A device for circumcising penis, according to claim 1, wherein the plurality of protrusions (201) comprises V-shaped protrusions.

9. A device for circumcising penis, according to claim 1, wherein each of the plurality of projections (301) has a stopper (105) to prevent the removal of the locking ring (300) from the primary ring (100).

10. A device for circumcising penis, according to claim 1, wherein the cover ring (400) is internally threaded.

11. A device for circumcising penis, according to claim 1, wherein the cover ring (400) has a plurality of teeth (401) circumferentially defined internally around said cover ring (400) to prevent the device from being disassembled.

12. A device for circumcising penis, according to claim 1, wherein the cover ring (400) includes the plurality of teeth (401) circumferentially defined externally around said cover ring (400) to prevent the device from being disassembled.

13. A device for circumcising penis, according to claim 1, wherein the cover ring (400) is screwed on to the primary ring (100) to constrict blood flow of the foreskin.

14. A device for circumcising penis, according to claim 1, wherein the primary ring (100), the locking ring (300), and the cover ring. (400) remain on the penis for at least five days after a procedure for circumcising the penis.

15. A device for circumcising penis, according to claim 1, wherein the foreskin, the primary ring (100), the locking ring (300), and the cover ring (400) fall off from penis after a minimum of five days after a procedure for circumcising the penis.
